(19) United States
(12) Patent Application Publication
(10) Pub. No.: US 2018/0353114 A1
(43) Pub. Date: Dec. 13, 2018

(54) Biological detection system

(71) Applicant: Macquarie University, Macquarie Park, NSW (AU)

(72) Inventors: Ewa Patrick GOLDYS, Kingsford, NSW (AU); Guozhen LIU, North Epping, NSW (AU)

(73) Assignee: Macquarie University, Macquarie Park, NSW (AU)

(21) Appl. No.: 16/061,218

(22) PCT Filed: Dec. 16, 2016
(86) PCT No.: PCT/AU2016/000401

§ 371 Date: Jun. 11, 2018

(30) Foreign Application Priority Data
Dec. 18, 2015 (AU) 2015905278

(51) Int. Cl. A61B 5/145; A61B 5/1473

(52) U.S. Cl. CPC A61B 5/14546; B82Y 30/00; A61B 5/1473

(57) ABSTRACT

A biological capture device including an elongate member configured to be inserted into a region of a body of a subject and having capture region on the surface of the elongate member containing a capture ligand capable of binding to a target biological material when the device is in the body, and methods of use of the device to detect and measure target biological materials in a body.

### TECHNICAL FIELD

The technology relates to biological capture device and system to allow in vivo capture and determination of a site of production of a target biological material in a subject.

### BACKGROUND

Methods for detecting biological materials outside of the body in biological samples are well known and used extensively. Detecting biological materials in the body and determining actual site of production would be useful in a number of biological research and clinical situations.

Cytokines are small secreted proteins (˜6-70 kDa) released by cells have a specific effect on the interactions and communications between cells. Certain pro-inflammatory cytokines such as IL-1β, IL-6, and TNF-α in spinal cord, dorsal root ganglion (DRG), injured nerve or skin are known to be involved in the process of pathological pain and with the generation of abnormal spontaneous activity from injured nerve fibres or compressed/inflamed DRG neurons. Thus in order to understand the role of the immune system in pain or cell-to-cell signalling, it would be useful to be able to monitor the concentration of the localized pro-inflammatory cytokine in the spinal cord or other regions of the body.

The present inventors have developed a biological capture device and system to allow in vivo capture and determination of a site of production and the level of production of a target biological material in a subject.

### DISCLOSURE OF INVENTION

In a first aspect, there is provided a biological capture device to capture of a target biological material in a subject, the device comprising:
an elongate member configured to be inserted into a region of a body of a subject; and
capture region on a surface of the elongate member, the capture region containing a capture ligand capable of binding to a target biological material when the device is in the body of a subject.

The elongate member may be any suitable material that can be inserted into the body and be adapted to contain the capture region. In an embodiment the elongate member is a cable, wire, rod, tube, glass optical fibre, plastic optical fibre or a strip made of a suitable material such as silica, gold, stainless steel or the like.

In an embodiment the elongate member is an optical fibre treated to allow coating with the capture ligand on its surface. Examples of suitable optical fibres include glass multimode optical fibres such as a 50/125 m multimode graded index optical fibre specified in Recommendation ITU-T G.651, or any other glass fibre used by the telecommunication industry to send optical signals. The optical fibre cladding can be removed prior to the formation of the capture region by a stripper such as solvent or other chemical or physical treatment.

The subject can be an animal, laboratory animal or a human.

The region of the body can be suitable region such as spinal cord, reproductive tract, urethra; fallopian tube, gastrointestinal track or a surgery field.

The capture region can be along the length of the elongate member that is to be positioned in the body. The capture region can be continuous or positioned on designated areas along the elongate member.

In an embodiment the capture ligand is an antibody or antibody fragment capable of binding to a target biological material.

In an embodiment the capture ligand is an antigen capable of binding to a target biological material.

The target biological material can be cytokine, growth factor, antibody, cell surface antigen. Examples of cytokines include IL-6, IL-1β, IL-10, HMGB1, GDF-9, BMP-15, GM-CSF, MIF, MCP1, TNF-α, VEGF, MCSF, SDF-1, TGF-β, MD2, and Fractalkin.

The capture region can contain one type of capture ligand allow detection and measurement of one target biological material.

The capture region can contain two or more different capture ligands to allow detection and measurement of two or more target biological materials.

In an embodiment the capture ligand is an antibody or antibody fragment bound or associated with a nanomaterial. Suitable nanomaterials include gold nanoparticles, silver nanoparticles, graphene oxides, signal walled carbon nanotubes. Gold nanoparticles sourced from Sigma-Aldrich can be particularly suitable.

In one embodiment, the capture device is inserted into the body of the subject through a catheter having a lumen configured to receive the elongate member of the capture device. The catheter having porous regions configured to allow ingress of biological material into the lumen of the catheter.

Use of a catheter allows easy insertion of the capture device and assists with locating the position of the capture device in the body.

After removal of the elongate member from the body, any target biological material bound to the capture region of the member can be detected and amounts measured by a florescence meter.

In one embodiment fluorescent detection systems are used in an enzyme-linked immunosorbent assay (ELISA) to allow detection and quantitation of small amounts of target biological materials.

When the position of the elongate member in the body is known, the site of production of the target biological material can be determined by reference to where on the elongate member the biological material was bound.

In a further embodiment there is provided a catheter having a lumen configured to receive the capture device.

The catheter can be any suitable commercial catheter such as a test insertion and guide canulla for a spinal cord stimulator, or indwelling spinal drug pump.

In an embodiment the catheter having porous regions configured to allow ingress of biological material into the lumen at the porous regions of the catheter.

In an embodiment porous regions on the catheter are made by drilling holes along the wall of the catheter using laser technology such as high-pulse-rate UV lasers.

In a second aspect, there is provided a biological capture system comprising:
a biological capture device according to the first aspect;
a catheter having a lumen configured to receive the capture device; and
porous regions on the catheter configured to allow ingress of biological material into the lumen at the porous regions of the catheter.

In a third aspect, there is provided a method for detecting a target biological material in a subject, the method comprising:
positioning a biological capture device according to the first aspect to a region of a body of a subject;
allowing the capture device to remain in the body for sufficient time to capture any target biological material by the capture ligand;
removing the capture device from the body; and
detecting any bound target biological material on regions of the capture device.

In an embodiment the biological capture device is positioned in the body of the subject using a catheter having a lumen configured to receive the elongate member of the capture device.

In an embodiment the catheter having porous regions configured to allow ingress of biological material into the lumen at the porous regions of the catheter.

In an embodiment the method further comprises locating the position of the capture device in the body.

The position of the catheter in the body can be determined by standard techniques such as radiography, ultrasound, or visual inspection as appropriate.

In an embodiment, the method further comprises determining the site in the body that produced the target biological material by reference to the region of captured target biological material on the capture device relative to its position in the body.

In an embodiment the method may further comprise:
quantitating the amount of target biological material detected.

In a fourth aspect there is provided use of a biological capture device according to the first aspect to detect and measure a target biological material in the body of a subject.

In order that the present invention may be more clearly understood, preferred embodiments will be described with reference to the following drawings and examples.

### BRIEF DESCRIPTION OF THE DRAWINGS

FIG. 1 shows a scheme of a cytokine test strip.

FIG. 2 shows UV-vis of gold colloid solution.

FIG. 3 shows UV-vis of glass slides after step-wise modification with APTES, AuNP, and IL-6 antibody.

FIG. 4 shows fluorescence of glass slide after stepwise modification.

FIG. 5 shows the relationship between fluorescence intensity and the concentration of IL-6.

FIG. 6 shows calibration curve for IL-6.

FIG. 7 shows normalized fluorescence signals before and after BSA blocking in capture antibody modified fibres.

FIG. 8 shows calibration curve of IL-6 based on the fluorescence signal and IL-6 concentration obtained from optical fibre after its exposure to different concentration of IL-6 followed by the incubation of DG_SPIO_IL-6_Ab (27.5 μg mL⁻¹).

FIG. 9 shows relationship between fluorescence signal in the 200 μm and 450 μm windows along the imaged length of the fibre.

FIG. 10 shows relationship between the fluorescence signal and the response time of the immunosensing device for the determination of IL-6 with concentration of 25 pg mL⁻¹ and 200 pg mL⁻¹.

FIG. 11 shows the reproducibility of ten fabricated cytokine test strips for detection of 60 pg mL⁻¹ IL-6.

FIG. 12 shows image of the optical fibre based cytokine assay (with catheter) under bright field microscopy.

FIG. 13 shows IL-6 secretion profile of BV2 cells after LPS stimulation for the commercial ELISA and the fabricated spatial fibre based cytokine assay.

### DEFINITIONS

Throughout this specification, unless the context clearly requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, the term ‘consisting of’ means consisting only of.

Any discussion of documents, acts, materials, devices, articles or the like which has been included in the present specification is solely for the purpose of providing a context for the present technology. It is not to be taken as an admission that any or all of these matters form part of the prior art base or were common general knowledge in the field relevant to the present technology as it existed before the priority date of each claim of this specification.

Unless the context requires otherwise or specifically stated to the contrary, integers, steps, or elements of the technology recited herein as singular integers, steps or elements clearly encompass both singular and plural forms of the recited integers, steps or elements.

In the context of the present specification the terms ‘a’ and ‘an’ are used to refer to one or more than one (ie, at least one) of the grammatical object of the article. By way of example, reference to ‘an element’ means one element, or more than one element.

In the context of the present specification the term ‘about’ means that reference to a figure or value is not to be taken as an absolute figure or value, but includes margins of variation above or below the figure or value in line with what a skilled person would understand according to the art, including within typical margins of error or instrument limitation. In other words, use of the term ‘about’ is understood to refer to a range or approximation that a person or skilled in the art would consider to be equivalent to a recited value in the context of achieving the same function or result.

Those skilled in the art will appreciate that the technology described herein is susceptible to variations and modifications other than those specifically described. It is to be understood that the technology includes all such variations and modifications. For the avoidance of doubt, the technology also includes all of the steps, features, and compounds referred to or indicated in this specification, individually or collectively, and any and all combinations of any two or more of said steps, features and compounds.

### DESCRIPTION OF EMBODIMENTS

Cytokines are small secreted proteins (˜6-70 kDa) released by cells have a specific effect on the interactions and communications between cells. Certain pro-inflammatory cytokines such as IL-1β, IL-6, and TNF-α in spinal cord, dorsal root ganglion (DRG), injured nerve or skin are known to be involved in the process of pathological pain and with the generation of abnormal spontaneous activity from injured nerve fibres or compressed/inflamed DRG neurons. Thus in order to understand the role of the immune system in pain or cell-to-cell signalling, or multiple other health conditions which lead to immunoreactivity and the expression of cytokines it would be useful to be able to monitor the concentration of the localized pro-inflammatory cytokine in the spinal cord, or in other location of the body, such as the reproductive tract, cancer stroma etc.

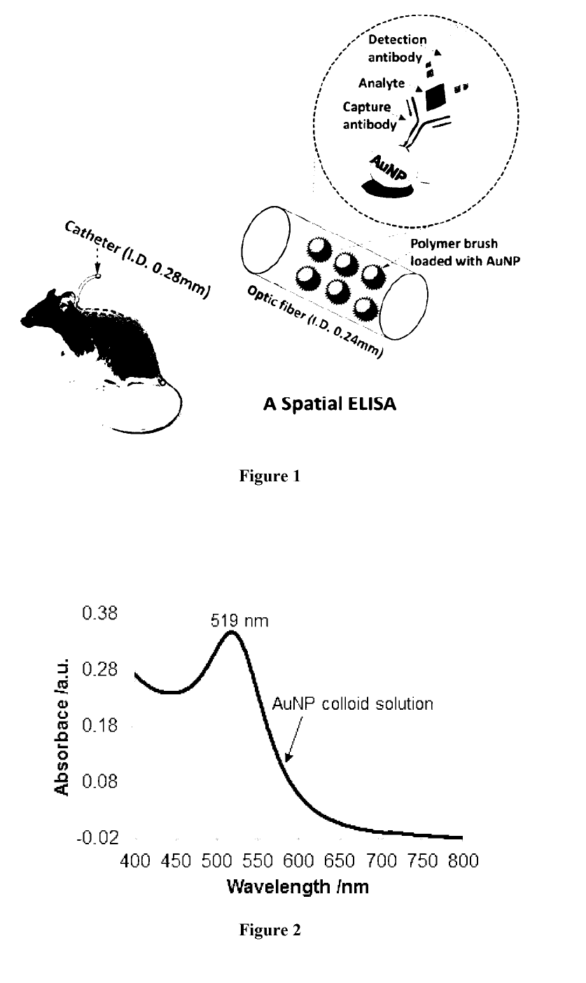

An in vivo device (FIG. 1) was designed which can be provided to the spinal cord of an animal such as a mouse for monitoring the localized cytokine concentration. A capture device was made from de-cladded optical fibre modified with a layer of polymer brush loaded with gold nanoparticles (AuNP) which was functionalized with capture antibody to form the sensing interface. This stripped optical fibre acted as a cytokine test strip. The capture device was then able to be introduced into a catheter with holes drilled along its length to enable fluid exchange between the outside and inside of the catheter.

The catheter may be inserted into the target location inside of the body such as the spinal cord of a subject with the capture device already inside or without the capture device. If the catheter is inserted during surgery without the capture device can be introduced at a later stage, when desired. The capture device can be removed from a catheter at any stage and a new capture device can be then reintroduced for a second or subsequent measurements. The removed capture device carries bound cytokines, so it forms a “cytokine test strip”. The ability to reintroduce the capture device is an advantage of the detection system.

After exposure of the capture device to cytokine-containing material (spinal cord) for a desirable period of time, the capture device is removed from the catheter and exposed to the detection antibody solution followed by quantification of cytokines based on the intensity of fluorescence. This variant of spatial ELISA was successfully used for the detection of cytokine IL-6 with the sensitivity of 1 pg mL-1, and showed high specificity to IL-6 where the matrix effect from the body fluid was excluded with the presence of polymer brush. This in vivo device is also useful to carry out multiplex cytokine monitoring in the body (such as the spinal cord by attaching different fluorophores onto detection antibodies). This technology offers the potential to understand the mechanisms underlying pathological pain and future effective pain therapies and multiple other health conditions. The technology is also applicable to reproductive health.

### EXPERIMENTAL

### Capture Device

In an embodiment the capture device is formed from an optical fibre obtained commercially such as Corning Infinicor 300 OM1-62.5/125/250 urn Multimode Optical Fiber.

Other suitable elongate members for the capture device include any “bulk” optical fibre comprising a single glass fibre inside of plastic cladding (i.e not an optical fibre cable which is an ensemble of several fibres) or a suitable plastic fibre with the surface able to be coated with the capture ligand.

### Capture Ligand

In an embodiment the capture ligand was made from gold nanoparticles produced by Sigma-Aldrich.

Other suitable capture ligands for the capture device include silver nanoparticles, graphene oxides, signal walled carbon nanotubes.

### Catheter

In an embodiment a catheter obtained from Becton Dickinson Intramedic Clay Adams Brand Number 427401 internal diameter 0.28 mm, outer diameter 0.61 mm was used.

Other suitable catheters include a test insertion and guide cannula for a spinal cord stimulator, or indwelling spinal drug pump.

### Protocol for Drilling Holes in Catheter by Laser

The protocol for drilling holes in catheter is detailed in Illy et al 2000. The samples were mounted on a closed-loop 2-axis computer-controlled positioning system (Physik Instrumente) giving linear write speeds from <1 mm/s up to 12.5 mm/s. Linear write speeds of up to 200 mm/s were achieved using a rotary motor to spin the sample for trepanning of holes. The depth of machined (rectangular cross-section) grooves was measured using a profilometer (Sloan Dektak 3030) for the samples and an optical microscope for the thicker PMMA samples. The uncertainties in groove depth were approximately 3% for the profilometer and 5% for the microscope. The power used for catheter sample is 10 mW with 10 s exposure/30 μm in and 6 μm out.

### Synthesis of 4-carboxyphenyl diazonium tetrafluoroborate

The corresponding aniline (0.01 mol) was dissolved by warming into 3 mL of concentrated hydrochloric acid (10 M) and 12 mL of water. A precipitate was obtained by cooling down to 0° C. in an ice/acetone bath. This precipitate disappeared after slow addition of a solution containing 0.752 g of sodium nitrite (0.011 mol) in 2 mL of water with vigorous stirring. The solution was filtered, and 1.48 g (0.013 mol) of sodium tetrafluoroborate was added with stirring. The thick slurry was cooled below 0° C. in an ice/acetone bath, filtered by suction, and washed with a cooled 5% NaBF4 solution to remove traces of acid and then washed with cold ether. The powder was dried in vacuum. Recrystallisation was carried out with a mixture of acetonitrile and cold ether to give the expected product. The diazonium salt was kept in a desiccator, at 4° C. over phosphorus pentaoxide.

Modification of IL-6 Antibody with 4-Carboxyphenyl Diazonium Tetrafluoroborate

10 mg of 4-carboxyphenyl diazonium tetrafluoroborate, 20.6 mg EDC, and 11.5 mg NHS were mixed in 1 mL of water for 10 min. From this solution 10 μL was added to 200 μL of 5 μg/ml anti-IL-6 antibody solution in carbonate buffer, pH 11, and was allowed to react for 2 hours at room temperature. The solution was then run through a 100,000 MW cut off Centricon centrifugal filter (Millipore, Billerica, Mass.) for 5 min at 4000 rpm and washed twice with 200 μL of water for 5 min at 4000 rpm before being brought up to a final volume of 200 μL with water.

Poly(Oligo(Ethylene Glycol)Methacrylate (POEGMA, Mn=360,300 mg mL⁻¹) Brush Growth

De-cladded optical fibre was immerse in a piranha solution (H₂SO₄ 95% and H₂O₂ 30% at a volume ratio of 4:1) for 30 min in order to clean the optical fibre and to form hydroxyl groups on it. After rinsing with deionized H₂O and drying under N₂ stream, the cleaned optical fibre was immersed in aminopropyltriethoxysilane (5% v/v) in toluene for 2 h to form amine terminated self-assembled monolayers on it. The fibre was then rinsed with toluene and ethanol to remove the unbound monomers from the surface. Then the substrates were immersed in a solution of bromoisobutyryl bromide (1%) and triethylamine (1%) in dichloromethane for 1 h, rinsed with dichloromethane and ethanol and blown dry with N₂. Polymerization was performed by immersing the substrates at room temperature in a degassed solution of Cu(I)Br (143 mg), 2,2′-bipyridyl (312 mg) and OEGMA (8.6 mL) in methanol (12 mL) and deionized water (3 mL) under nitrogen purge for a stipulated duration. Finally the substrates were removed, thoroughly rinsed with methanol and blown dry with N₂.

Nanoparticle Incorporation into POEGMA

The optical fibre had been earlier modified with POEGMA were subsequently immersed in 3 mL of AuNP (10 nm) solution and left overnight. Upon removal, they were copiously rinsed with deionized H₂O. They were then rinsed 3 times in deionized H₂O under orbital shaking at 120 rpm for 2 min each and finally blown dry with N₂.

Suitable methods for preparing nanoparticles containing antibodies and detecting bound biological materials to nanoparticles can be found in Ferhan et al 2013.

### Attachment of Capture IL-6 Antibody to the AuNP Incorporated POEGMA

A 200 μL drop of 40 μg/mL diazonium-modified anti-IL-6 capture antibody in 5 mM HCl solution was placed on the glass slide, covering all surfaces, which was left to react at room temperature for 2 h. The surface was then washed with water and briefly dried under a stream of nitrogen. The formed sensing interfaces were exposure to the analyte of IL-6 for 5 min. Then the surface was rinsed with lots of water and blow drying under nitrogen. Finally the sensing interface was immersed in the MPSi_anti-IL-6_Ab solution for 1 h followed by rinsing with lots of water and drying under N₂.

Suitable detection systems are set out in Harper at al 2007. In this system capture antibodies were attached to the sensing interfaces by aryldiazonium salt chemistry through forming Au—C bonds, which is beneficial to the stability of the designed device. [

Results of experiments regarding preparing the biological capture device and binding and detection of target biological materials are shown in FIGS. 2 to 4.

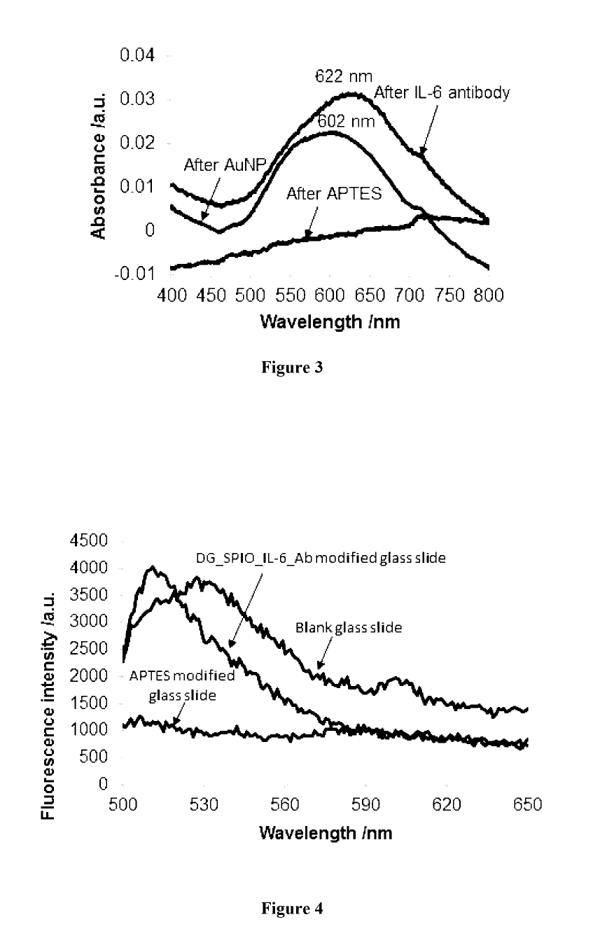

The self-assembled AuNPs deposited on a glass cover slip were characterized by UV-vis spectrophotometry (FIG. 2 and FIG. 3). The spectrum of the AuNP colloid solution has a characteristic plasmon peak at 519 nm. The peak is absent for the glass surface after modification of APTES, however, a similar feature appears at 602 nm after modification of glass with AuNP, which confirms the successful attachment of AuNP. This spectral characteristics indicates that the Au colloids self-assembled on glass are close enough to affect the coupling of plasmons of individual particles resulting in an increased absorbance at wavelengths >600 nm when compared to that of the original AuNP in solution. The plasmon peak had a further redshift (622 nm) after the attachment of IL-6 capture antibody due to the change of the surrounding environment of AuNPs.

Fluorimetry was used to monitor surface modifications of the glass surface after the attachment of detection antibodies (FIG. 4). The background fluorescence signal for the blank glass surface was observed around 530 nm and 600 nm respectively; it disappeared after modification of the glass surface with APTES, due to the formation of a layer of amine groups on the surface. After stepwise further modification of the glass surface with 6-mercaptohexanoic acid, AuNP, incubation with the anti-IL-6 capture antibody followed by the incubation with IL-6 (100 pg mL-1) and the fluorescent detection antibody (DG_SPIO_IL-6_Ab), the characteristic fluorescence peak of the Dragon Green beads appeared at about 518 nm, indicating a successful attachment of the detection antibody. Thus this fabricated system is capable of detection of IL-6. The successfully attachment of AuNP was further confirmed by the SEM images of the AuNP modified glass slides.

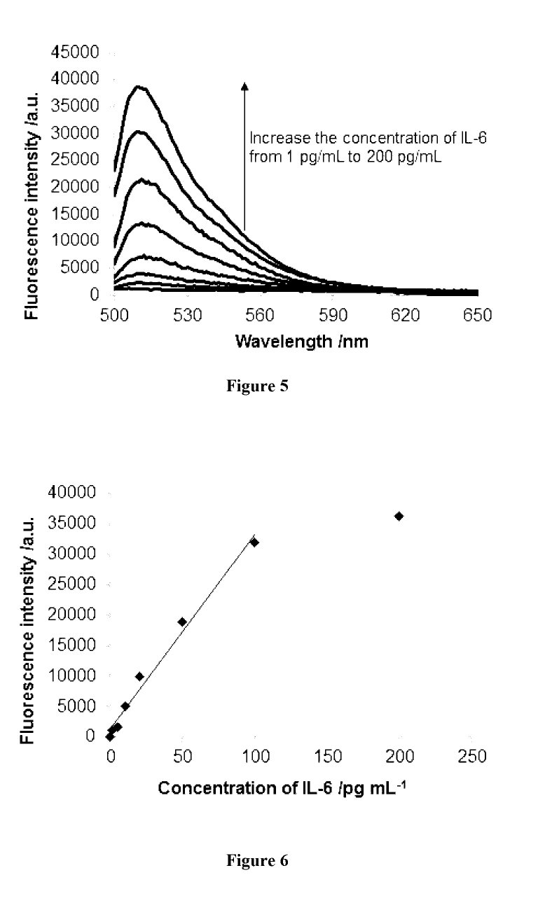

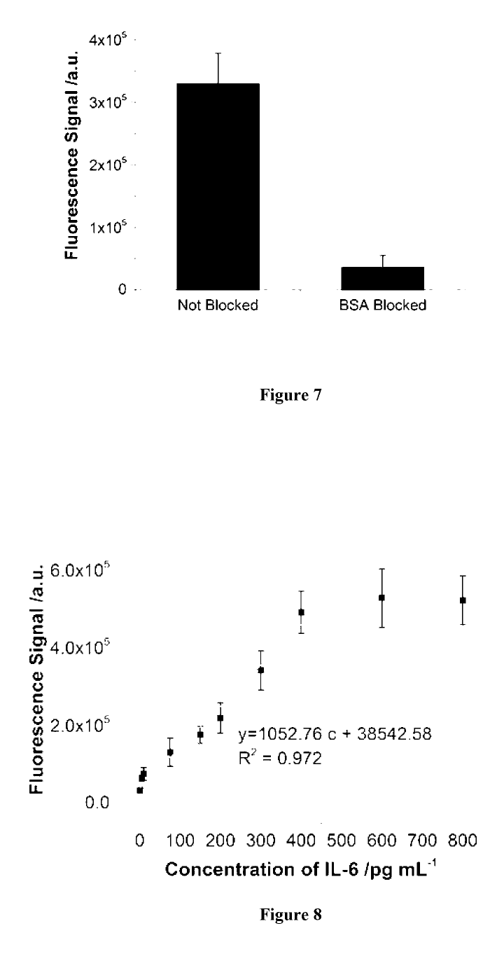

The fabricated sensing interface on the glass cover slip was used to detect IL-6 at different concentrations. FIG. 5 and FIG. 6 show the relationship between fluorescence intensity and the concentration of IL-6, and the fluorescence signal of the detection antibodies conjugated to Dragon Green beads increased linearly with the concentration of IL-6. The calibration curve of this sensing interface for IL-6 is plotted in FIG. 7. The lowest detectable concentration was 1 pg mL-1 with the linear range of 1-100 pg mL-1 within the physiological concentration range of IL-6 in the body. Thus this assay can be used to quantify the IL-6 concentration in vitro. This immunosensor scheme has been further applied to the optical fibre, as described in the following sections.

### Performance of the Fabricated Sensor on Optical Fibre Surface

After verifying the performance of the cytokine capture surface on glass slides an identical capture surface was fabricated on a glass fibre 62.5 μm in diameter. In order to quantify the fluorescence signal reporting on the presence of analyte molecules captured on fibre surface a tailored approach was developed. Drawing on the capability of laser scanning confocal microscope to reject out of focus signal and its depth of field that is much smaller than the fibre diameter, multiple images at different axial planes (Z-stack), around 12.5 micrometer apart, to image the total visible fibre area were recorded. This Z-stack was further processed to select the maximum pixel value from each image. This maximum value was then assigned to the corresponding pixel (maximum Z-projection in Image-J). This final composite image produced from a Z-stack was taken as a representation of the total fluorescence signal of a section of the fibre and it was used for further quantification of the signal.

### Anti-Fouling Property of the Sensing Interface

The nonspecific protein adsorption of this sensing device was investigated using the blocking reagent bovine serum albumin (BSA) (FIG. 7). In order to bring down the nonspecific binding, the blocking reagent BSA was applied onto the sensing interface. In the absence of BSA blocking, a significant non-specific DG_SPIO_IL-6_Ab absorption was observed on the capture antibody-modified interface after the exposure to the detection antibody solution, likely to be due to physical adsorption of DG_SPIO_IL-6_Ab on surface defects. However, when the capture antibody-modified sensing interface was blocked with 1% BSA, only a few green dots were observed in the confocal image, suggesting negligible non-specific absorption (5 orders of magnitude lower than the signal for 100 pg mL⁻¹ IL-6). Such low non-specific adsorption is required to maximize the detection specificity of IL-6.

### Detection of IL-6 Using the Fabricated Cytokine Immunosensing Device

The capture antibody-modified sensing interface (with 1% BSA blocking) was further used for detection of IL-6 in phosphate buffered saline (PBS) solution. As found from the Z-stack maximum intensity projection images), the Dragon Green intensity increased with increasing IL-6 concentration, indicating that IL-6 could be quantified by integrating Dragon Green fluorescence. A linear relationship between the fluorescence intensity and the concentration of IL-6 in the range of 1 to 400 pg mL⁻¹ was obtained (FIG. 8) after quantification of the fluorescence signal by image J and Matlab software, which is within the physiologically relevant range (Liu et 2016). The lowest detectable concentration of IL-6 was 1 pg mL⁻¹, which is similar to that of an electrochemical immunosensor based on ferrocene-loaded porous polyelectrolyte nanoparticles as a label (1 pg mL⁻¹) (Li and Yang, 2011) The lowest detection limit is lower than the value reported in a recently developed liquid-gated field-effect transistor sensor based on horizontally aligned single-walled carbon nanotubes for detection of IL-6 (1.37 pg mL⁻¹), (Chen et al, 2016) and it is one order of magnitude lower than a fluorescence-based immunoassay (20 pg mL⁻¹) (Hun and Zhang, 2007). The application of AuNPs on the sensing interface for loading large amount of capture antibodies and the brightness of the nanoparticles labeled with detection antibody have contributed to the high sensitivity achieved. The reproducibility of the fabricated cytokine assay was evaluated by fabricating 10 separate pieces of optical fibres used for the detection of 60 pg mL⁻¹ IL-6. The relative standard deviation of these ten immunosensors was ±3.6%, indicating that the fabricated assay was closely reproducible in tested conditions.

The feasibility of the fabricated cytokine assay for the spatially localised detection of IL-6 was studied by placing drops (˜1 μL) of the serum sample containing 10 pg mL⁻¹ and 200 pg mL⁻¹ IL-6 onto various locations of the fabricated optical fibre surfaces, respectively, followed by incubation with the detection antibody. Subsequently, the optical fibre exposed to two different concentrations of IL-6 in two close locations was imaged (3 mm length in total) and signal quantification carried out. The intensity of the green dots representing the Dragon Green fluorescent labels were quantified by integrating over a spatial window of specific width (typically 100-500 μm) using Image J and Matlab software. This spatial window was chosen to ensure enough Dragon Green beans are imaged for the fluorescence quantification, even for the lowest cytokine concentration. The width of the spatial window is one of the factors determining the achievable spatial resolution. The fluorescence in the fibre area exposed to 200 pg mL⁻¹ IL-6 was found to be significantly higher (5 times) than that with 10 pg mL⁻¹ IL-6, suggesting that the fabricated sensing interface was capable to differentiate IL-6 at different concentrations from the sample volume of 1 μL. Thus, the cytokine immunosensing device developed requires minimal sample consumption and it offers excellent assay performance making it highly suitable for analyzing biomarkers and cytokines in precious biological samples. Moreover, the fabricated fibre has the capability of spatially-resolved detection of localised IL-6 with the resolution in the order of 200 to 450 μm. It is understood that only the Olink Bioscience's Proseek protein assay enables sensitive detection and quantification of proteins in a 1 μL sample volume (Hjelm et al, 2011), but it does not offer spatial resolution.

The response time of the immunosensing device for the detections of IL-6 at the concentrations of 25 pg mL⁻¹ and 200 pg mL⁻¹, respectively were also determined. The fluorescence signal of the SPIO-Ab increased dramatically with increasing incubation time for IL-6 (200 pg mL⁻¹) and it saturated around 30 min. In the case of lower concentration of IL-6 (25 pg mL⁻¹), the fluorescence signal increased about one order of magnitude more slowly than that for the high concentration of IL-6 (200 pg mL⁻¹). This more rapid transition towards the equilibrium is as expected by the basic laws of chemical kinetics (Reverberi and Reverberi 2007). For the concentration of 200 pg mL⁻¹, a measurable signal increase was observed after 5 minutes incubation in the cytokine-containing medium.

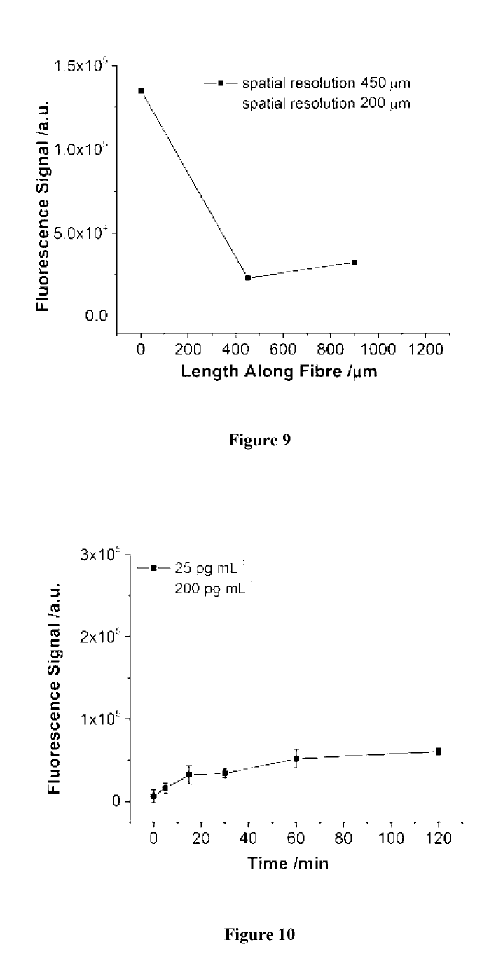

Spatially localized cytokine detection experiments were carried out using a device according to the present technology. An amount of 1 μL of serum containing 10 pg mL⁻¹ and 200 pg mL⁻¹ IL-6 was simultaneously placed on the fabricated sensing interface, respectively, followed by incubation with detection antibody and signal quantification. FIG. 9 shows the relationship between fluorescence signal in the 200 and 450 μm windows along the imaged length of the fibre. FIG. 10 shows the relationship between the fluorescence signal and the response time of the immunosensing device for the determination of IL-6 with the concentration of 25 pg mL⁻¹ and 200 pg mL⁻¹.

### Reproducibility of the Fabricated Cytokine Test Strip

The reproducibility of the fabricated cytokine test strip was evaluated by fabricating 10 optical fibres used for the detection of 60 pg mL⁻¹ IL-6 (FIG. 11). The relative standard deviation of these ten immunosensors was ±3.6%, indicating that the fabricated assay was closely reproducible in tested conditions.

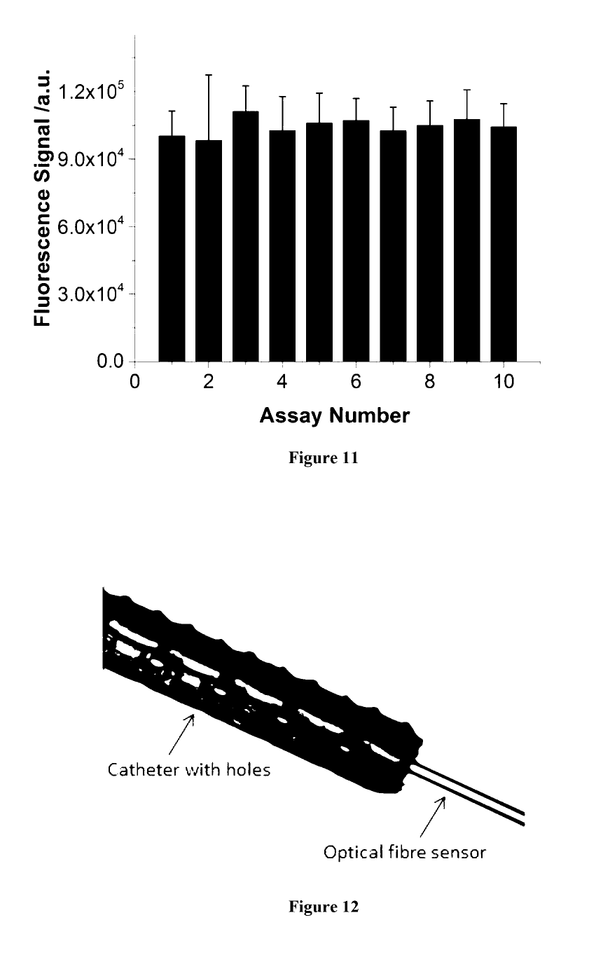

Finally, the optical fibre sensor with the catheter on was applied for the detection of IL-6 secreted by live BV2 cells. FIG. 12 shows the device which was used for the measurements in the medium. The concentration of IL-6 secreted into the medium increased with the LPS stimulation time and the maximum concentration was obtained after 6 h LPS stimulation. Similar IL-6 secretion pattern for BV2 cells was obtained by conventional ELISA but the lowest detection limit of our fibre device (1 pg mL⁻¹) was one order of magnitude lower than the conventional ELISA Kit from R&D System for IL-6 (10 pg mL⁻¹). In these experiments no media needed to be removed from the culture, instead repeated sampling can be achieved by replacing the fibre capture device. Therefore the cytokine assay device was capable to monitor cytokines ex-vivo.

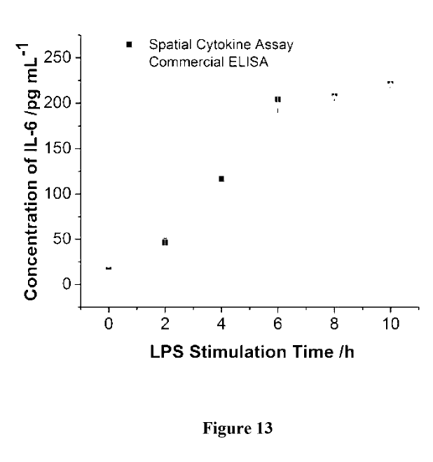

FIG. 13 shows the IL-6 secretion profile of BV2 cells after LPS stimulation for the commercial ELISA and the herein fabricated spatial fibre based cytokine assay.

A sensitive cytokine assay was fabricated and characterised based on an optical fibre, which could be used for monitoring the localised cytokine concentration ex-vivo. A spatially-resolved ELISA sandwich assay was built on the optical fibre surface so that the fibre could be inserted into a perforated catheter. After exposure of the device to the cytokine-containing solution for a period of time, the optical fibre forming a cytokine test strip was removed from the catheter which could be inserted into the body. The fibre was then exposed to the solution of the detection antibody conjugated to fluorescent Dragon Green beads and washed followed by quantification of cytokines based on the intensity of fluorescence by laser scanning microscopy. This variant of spatial ELISA was successfully used for the detection of cytokine IL-6 with the low limit of detection of 1 pg mL⁻¹ and sample volume of 1 μL, and it showed high specificity to IL-6. The sensor interface was stable for up to 9 days in PBS solution, and it was capable of detecting localised IL-6 secreted by BV2 cells with liposaccharide stimulation. This technology provides a new strategy for monitoring spatially varying concentration of cell secreting products, and it has the potential to be developed as point-of-care device for multiple health conditions.

### DISCUSSION

Cytokines are small proteins secreted from cells and can be indicators of the functional status of the human immune system and involved in dynamic immune reactions. Cytokines are biomarkers for many diseases and have very low concentration (pM range) in serum.

Current approaches to detect cytokines are all ex-vivo so the present invention provides a useful alternative for in vivo detection.

Advantages of the detection system include high sensitivity, biocompatibility, strong chemical stability, capability of resisting non-specific protein adsorption, relatively inexpensive to manufacture.

Super bright fluorescent detection enables measurements with a simple microscope. More sensitive particles allow for point-of-care disposable test strip to be developed.

In vivo diagnostics allow therapeutic drug testing, early disease detection, and sensor technologies for biological and chemical detection.

A number of multiplex cytokine assays and platforms exist. Cytokines (average) can be detected in body fluids by—ELISA (not very sensitive) multiplex Luminexbead assay (expensive).

### USES

### Fertility

Help determine optimal uterine cytokine environment for pregnancy establishment, diagnose endometriosis lesions, semen cytokine analysis for male fertility testing, mastitis testing in both animals and humans.

Infertility is a condition that has enormous psychological consequences for couples—over 2 million babies are born world-wide each year by assisted reproductive technologies. Enhancing the predictive capacity for couples undergoing ART to achieve pregnancy is significant.

### Trauma Medicine and Neuroscience

Detect local cytokine expression in lab animals, diagnose seriousness of spinal injury, and detect local cytokine expression in regions.

There is a limited number of methods to assess the seriousness of spinal injury. Accurate diagnosis is key to effective treatment.

The device may be used to detect post-injury inflammation caused by traumatic brain injury or spinal cord injury.

While doing in vivo diagnostics, the device may allow therapeutic drug testing, early disease detection, and sensor technologies for biological and chemical detections.

The device may be used as the point-of-care disposable test strip to monitor multiple health conditions in clinical situations.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

Illy, E. K.; Brown, D. J.; Withford, M. J.; Piper, J. A. In Optimization of trepanning strategies for micromachining of polymers with high-pulse-rate UV lasers, Advanced High-Power Lasers and Applications, International Society for Optics and Photonics: 2000; pp 608-616.
Ferhan, A. R.; Guo, L. H.; Zhou, X. D.; Chen, P.; Hong, S., Kim, D. Solid-phase colorimetric sensor based on gold nanoparticles-loaded polymer brushes: lead detetion as a case study, Analytical Chemistry: 2013; pp 4094-4099
Harper, J. C.; Polsky, R.; Wheeler, D. R.; Dirk, S. M.; Brozik, S. M. Selective Immobilization of DNA and Antibody Probes on ElectrodeArrays: Simultaneous Electrochemical Detection of DNA and Protein on a Single Platform, Langmuir, 2007; 23(16), pp 8285-8287
Liu, G. Z.; Qi, M.; Hutchinson, M. R.; Yang, G. F.; Goldys, E. M., Recent advances in cytokine detection by immunosensing. Biosens. Bioelectron. 2016, 79, 810-821.
Li, T.; Yang, M., Electrochemical sensor utilizing ferrocene loaded porous polyelectrolyte nanoparticles as label for the detection of protein biomarker IL-6. Sens. Actuators B: Chem. 2011, 158 (1), 361-365.
Chen, H.; Choo, T. K.; Huang, J.; Wang, Y.; Liu, Y.; Platt, M.; Palaniappan, A.; Liedberg, B.; Tok, A. I. Y., Label-free electronic detection of interleukin-6 using horizontally aligned carbon nanotubes. Mater. Des. 2016, 90, 852-857.
Hun, X.; Zhang, Z., Functionalized fluorescent core-shell nanoparticles used as a fluorescent labels in fluoroimmunoassay for IL-6. Biosens. Bioelectron. 2007, 22 (11), 2743-2748.
Hjelm, F.; Tran, B.; Fredriksson, S., Sensitive detection of cytokines in 1-[mu]| serum samples using Proseek [reg]. Nat. Methods 2011, 8 (9), iii-iv.
Reverberi, R.; Reverberi, L., Factors affecting the antigen-antibody reaction. Blood Transfusion 2007, 5 (4), 227.

1. A biological capture device to capture of a target biological material in a subject, the device comprising:
an elongate member configured to be inserted into a region of the body of a subject; and
capture region on a surface of the elongate member, the capture region containing a capture ligand capable of binding to a target biological material when the device is in the body of a subject.

2. The device according to claim 1 wherein the elongate member is a cable, wire, rod, tube, optical fibre, or strip of a silica, gold, or stainless steel.

3. The device according to claim 2 wherein the elongate member is an optical or glass fibre.

4. The device according to claim 3 wherein the optical or glass fibre is treated to allow coating with the capture ligand on its surface.

5. The device according to claim 1 wherein the capture region is on a region of the elongate member that is to be positioned in the body.

6. The device according to claim 5 wherein the capture region is continuous or positioned on designated areas along the elongate member.

7. The device according to claim 1 wherein the capture region comprises two or more different capture ligands to allow detection of two or more target biological materials.

8. The device according to claim 1 wherein the target biological material is selected from the group consisting of cytokine, growth factor, antibody, and cell surface antigen.

9. The device according to claim 8 wherein the cytokine is selected from the group consisting of IL-6, IL-1β, IL-10, HMGB1, GDF-9, BMP-15, GM-CSF, MIF, MCP1, TNF-α, VEGF, MCSF, SDF-1, TGF-β, MD2, and Fractalkin.

10. The device according to claim 1 wherein the capture ligand is an antibody or antibody fragment capable of binding to the target biological material.

11. The device according to claim 10 wherein the antibody or antibody fragment is bound or associated with a nanomaterial.

12. The device according to claim 11 wherein the nanomaterial comprises gold nanoparticles, silver nanoparticles, graphene oxides, or signal walled carbon nanotubes.

13. The device according to claim 1 further comprising a catheter having a lumen configured to receive the elongate member of the capture device.

14. The device according to claim 13 wherein the catheter comprises porous regions configured to allow ingress of biological material into the lumen of the catheter.

15. A method for detecting a target biological material in a subject, the method comprising:
positioning a biological capture device according to claim 1 to a region of a body of a subject;
allowing the capture device to remain in the body for sufficient time to capture any target biological material by the capture ligand;
removing the capture device from the body; and
detecting any bound target biological material on regions of the capture device.

16. The method according to claim 15 wherein the device is positioned in the body of the subject using a catheter having a lumen configured to receive the elongate member of the capture device.

17. The method according to claim 16 wherein the catheter comprises porous regions configured to allow ingress of biological material into the lumen of the catheter.

18. The method according to claim 15 further comprising locating the position of the capture device in the body; and
determining the site in the body that produced the target biological material by reference to the region of captured target biological material on the capture device relative to its position in the body.

19. The method according to claim 15 further comprising:
quantitating the amount of target biological material detected.

20. Use of a biological capture device according to claim 1 to detect and measure a target biological material in the body of a subject.
